(19) United States
(12) Patent Application Publication
(10) Pub. No.: US 2013/0030235 A1
(43) Pub. Date: Jan. 31, 2013

(54) METHOD FOR DESULFURIZING OLEFIN-CONTAINING CHARGE MATERIAL BY CONTROLLING THE OLEFIN CONTENT

(71) Applicants: Thilo Von Trotha, Selm (DE); Frank Urner, Dortmund (DE)

(72) Inventors: Thilo Von Trotha, Selm (DE); Frank Urner, Dortmund (DE)

(73) Assignee: THYSSENKRUPP UHDE GMBH, Dortmund (DE)

(21) Appl. No.: 13/382,822

(22) PCT Filed: Jul. 7, 2010
(86) PCT No.: PCT/EP2010/004092

§ 371 Date: Jun. 27, 2012

(30) Foreign Application Priority Data
Jul. 10, 2009 (DE) 10 2009 032 802.5

(51) Int. Cl. C07C 7/163

(52) U.S. Cl. 585/809

(57) ABSTRACT

A process and an apparatus for desulphurisation of a feed stream containing olefins and hydrogen which is split into at least two feed streams. The first feed stream is introduced into the reactor and reaches a first catalyst bed and is heated by the hydrogenation reaction. Downstream the second feed stream is supplied which will cool down the reaction gas which can then be passed through a second catalyst bed. The content the feed streams can be controlled by adding olefins or dilution gas. The reaction will generate a product gas which will basically contain hydrogen sulphide as a sulphur compound. The temperature of the catalysts and of the gas flow is controlled via the olefin content in the feed streams. The higher the olefin content in the feed stream, the more intense the heating of the gas flow by the hydrogenation heat in the subsequent catalyst bed.

The invention relates to a process for the hydrogenation of material flows containing olefins and sulphur as commonly occurring in crude oil refineries. By the process according to the invention the sulphur compounds contained in these flows are hydrogenated in a reactor to achieve a complete or partly conversion into hydrogen sulphide and the olefins contained in these flows are hydrogenated to achieve a complete or partly conversion into alkanes. The process and especially the temperature distribution in the reactor are controlled by adjusting the olefin content in the feed streams supplied to the reactor. The invention also relates to a contrivance which serves to carry out the process and is suited to implement the mentioned process steps.

DE 102007059243 A1 describes a process for the hydrogenation of olefin-containing material flows which incorporate organic sulphur compounds and are converted into hydrogen sulphide by hydrogenation. The hydrogenation serves to eliminate the sulphur compounds from the introduced material flow by removing the hydrogen sulphide from the product gas as obtained material mixture in a gas scrubbing process subsequent to the hydrogenation.

The feed streams are passed through the reactor, which is provided in gas flow direction with several successive catalyst beds serving to perform a consecutive hydrogenation. The feed streams typically consist in a gas or an evaporated liquid. Downstream of each catalyst bed there is a feed device for a further feed stream by which further feed stream can be introduced into the reactor gas flow. As the catalyst beds and the reactor gas flow heat up after each hydrogenation step, it is possible to control the temperature distribution inside the reactor via the distribution of the feed stream downstream of the individual catalyst beds. By adding fresh feed stream downstream of the respective catalyst bed, the feed stream will cool down again.

In this way it is possible to carry out the hydrogenation in a continually optimum temperature range. The catalyst can thus be kept at a temperature which ensures optimum range of application. This procedure will produce several material flows downstream of the individual catalyst beds. This may lead to different pressure conditions inside the reactor, which may pose a problem depending on the respective process type. It is therefore the aim to control the addition of the olefin downstream of the individual catalyst beds such that this addition is not performed via the flow rate control.

The invention achieves this aim by adding feed streams of precisely controlled olefin content. As the gas flow and the catalyst bed in the reactor are only heated by the reaction heat of the olefin hydrogenation reaction, the temperature distribution in the reactor can be controlled by adding feed streams of different olefin contents. Here, a feed stream always signifies a gaseous material flow.

Especially claimed is a process for the hydrodesulphurisation of olefin-containing feedstocks with the aid of a feed stream containing hydrogen, in which
a gaseous feed stream containing olefins and hydrogen is passed through a reactor provided with a catalyst suitable for hydrodesulphurisation, and the organic sulphur compounds and olefins contained in the feed stream incorporating olefins and hydrogen are hydrogenated completely or partly into hydrogen sulphide and alkanes, and
the olefin-containing feed stream is split prior to being introduced into the reactor so that at least two feed streams are formed, and
the first feed stream is passed by means of suitable devices via the reactor head and through a catalyst bed inside the reactor containing a partial amount of a catalyst suitable for hydrodesulphurisation, and
a second feed stream is added into the reactor laterally downstream of the first catalyst bed and to the reaction mixture heated by the first hydrogenation, and the gas flow thus obtained is passed through a second catalyst bed inside the reactor,

and which is characterised in that
the content of olefins in at least one feed stream can be controlled by separately adding olefins or dilution gas to the individual feed streams,
the temperature inside the reactor being controlled by adjusting the content of olefins in at least one feed stream.

Part of the total olefin amount is supplied via the head of the reactor. The temperature at the reactor head is usually approx. 300° C., which is well suited to carry out the hydrogenation reaction. The content of olefins in the first olefin-containing feed stream can advantageously be controlled by adding a dilution stream lean in or free of olefins or both dilution streams to the first feed stream. In this way an olefin-containing feed stream is produced.

The olefin-lean and the olefin-free feed stream may be added in a mixture with the option to either add them separately in two individually controlled streams or in premixed condition. By adding these two material mixtures as dilution streams it is possible to adjust the desired content of olefins in the feed stream and also to control the temperature in the reactor. Depending on the desired process mode it is also possible to introduce another material flow containing a gas lean in or free of olefins. Thus it is possible to further dilute the feed stream. It is also possible to increase the content of olefins in the first feed stream by separate addition of a material flow rich in olefins into the first feed stream. In principle, the first feed stream introduced contains olefins already.

According to an embodiment of the invention a material flow which is lean in olefins and a material flow which is free of olefins are added as dilution streams to the first feed stream. In this way it is possible to control the hydrogenation via the olefin content in this flow such that it produces a precisely defined amount of heat. This serves to adjust the temperature downstream of the first catalyst bed such that mixing it with the second feed stream will produce exactly the temperature that is required for the passage through the second catalyst bed.

If it seems to be necessary, it is possible to add an olefin-rich material flow to the feed stream in order to increase the olefin content in the first feed stream. This may be done on a temporary or a permanent basis. The olefin-rich material flow can be added separately or in premixed condition with another material flow. Finally it is possible to add an olefin-free, olefin-lean and olefin-rich material flow separately to the first feed stream so to control the olefin content in the first feed stream. Although the addition is preferably performed separately, it is also possible to add these material flows in premixed condition. The pre-mixture can be implemented in any mixing combination and ratio desired.

The reactor may be provided with more than two catalyst beds. In another embodiment of the invention the material flow obtained from the reaction is passed through a third catalyst bed, which will heat the bed and the passing gas flow. This means that downstream of the second catalyst bed a third feed stream is supplied into the reactor laterally downstream of the second catalyst bed to the material flow which has been heated by the second hydrogenation and that the gas flow for hydrogenation first passes the second catalyst bed and then the third catalyst bed.

An embodiment of the invention provides in an exemplary fashion that downstream of the second catalyst bed a third feed stream is fed into the reactor laterally downstream of the second catalyst bed to the material flow heated by the second hydrogenation, and the material flow to be hydrogenated passes the second catalyst bed first and then a third catalyst bed. It is possible to pass the material flow, which has been sent through the third partial amount of the hydrodesulphurisation catalyst, through one or several additional partial amounts of a hydrodesulphurisation catalyst and to introduce a further feed stream into the reactor laterally downstream of the catalyst beds.

To control the temperature distribution also in this third catalyst bed, a material flow lean in olefins and a material flow free of olefins are analogously fed into the supply line for the second feed stream downstream of the first catalyst bed. By the amounts of individual material flows admixed it is possible to also adjust the olefin content in this second feed stream. This makes it possible in turn to control the temperature of the third catalyst bed. Likewise it is possible according to an embodiment of the invention to additionally introduce a material flow rich in olefins into the reactor.

At last it is also possible to pass the gas flow through as many catalyst beds as desired. Downstream of each catalyst bed, a further material flow may be introduced laterally, being of an olefin content which allows to adjust the temperature of the subsequent hydrogenation in an optimum way. This means that after the material flow has been sent through the third partial amount of the hydrodesulphurisation catalyst it is passed through one or several additional partial amounts of a hydrodesulphurisation catalyst and a further feed stream is introduced into the reactor laterally downstream of the catalyst beds. It is possible to also supply a material flow lean in olefins or free of olefins to the respective feed stream to deplete the latter of olefins if this is required. In this way it is possible to adjust the olefin content of the respective feed stream by adding a material flow. The addition can be performed in the form of separate material flows which are lean in or free of olefins or in the form of premixed flows.

It is finally possible to increase the olefin content by adding an olefin-containing material flow. Such addition may optionally take place downstream of every catalyst bed desired. In general, however, this is not required. The addition of the mentioned material flows as dilution streams can be implemented in any mixing combination and ratio desired.

The olefin-free gas is preferably hydrogen, methane or a mixture of these substances. The olefin-lean gas is also preferably a gas which contains hydrogen or methane as main component or both. It is also possible, however, to add a different gas to the material flows supplied. This may, for instance, be alkanes or carbon dioxide. The olefin-rich, olefin-lean or the olefin-free material flow material may eventually be mixed in any form desired. Advantageously they also do not contain any undesired foreign gases.

The feed stream is preferably supplied via the head of the hydrogenation reaction reactor. The proportion of the gas supplied at the head is basically optional, preferably, however, it ranges between 1 and 99 percent by mass. Ideally the material flow of the gas supplied at the head ranges between 5 and 15 percent by mass. By the overall hydrogenation reaction it is possible to produce a feed stream which has a content of organic sulphur compounds of below 100 ppb. In a subsequent gas scrubbing process it is possible to remove the hydrogen sulphide so that a gas is obtained which is basically free of sulphur.

The feed stream as feed stream for the hydrodesulphurisation preferably contains light olefins which are in gaseous form at the operating temperature. These are preferably in the C-number range from 2 to 6. It is also possible, however, to use higher olefins which are in liquid form at the operating temperature or heavier hydrocarbons. These may also be in the higher C-number range. As feed stream it is basically possible to use all olefins that allow desulphurisation by hydrogenation and scrubbing.

The hydrogenation reaction is preferably carried out at a temperature of 150 to 500° C. Optimum temperatures range between 250 and 400° C. The feed stream is therefore preferably introduced into the reactor at a temperature of 200 to 400° C. With particularly suitable reaction parameters the feed stream is introduced into the reactor at a temperature of 250° C. to 350° C. The respective temperature in the reactor will then emerge from the prevailing reaction parameters. The introduction of an olefin-leaner feed stream in a suited place will cool down the reaction mixture. By controlling the reaction parameters via the olefin content of the feed streams it is much easier to control the pressure inside the reactor. In a favourable type of configuration the latter ranges between 0.1 and 10 MPa.

Heating of the feed stream up to the temperature required for the reaction may be done in any way desired. For this purpose, burners or steam heaters, for example, may be used. Heating of the feed stream will, however, preferably be implemented via heat exchangers in any place desired. As a heating agent the heated material flow in the reactor may be used. The heat exchangers may be used for heating in any place desired as, for example, on the individual feed streams. Heating may also be done on the material flows which are introduced into the feed streams. It may also be done on the feed stream which is introduced at the reactor head.

In an embodiment of the inventive process, the process for hydrodesulphurisation is followed by a gas scrubbing process or a separation of hydrogen sulphide, which may be of any type and at any point of the process desired. The process for hydrodesulphurisation may be followed, for example, by an adsorption process using a chemical adsorbent.

The invention also claims a contrivance which serves to run the process according to the invention. Especially claimed is a contrivance which is characterised in that
a pipeline conveying the feed stream splits the feed stream into at least two gas flows, and
the pipeline supplying the first feed stream via the head of a reactor fitted with several horizontally arranged catalyst beds, the reactor having at least two horizontally arranged catalyst beds, and
a second pipeline entering the reactor laterally is installed between the first and the second catalyst bed to introduce the second feed stream into the downward material flow so that the resulting material flow passes through the second catalyst bed, and
the pipelines for at least one feed stream are fitted with feed lines for material flows by which it is possible to control the olefin content in the feed stream.

These are feed lines which allow introducing an olefin-rich material flow into the respective feed stream. In such case, the olefin content in the feed stream increases and the temperature in the subsequent catalyst bed increases accordingly. These may also be feed lines for an olefin-lean or olefin-free material flow to reduce the olefin content in the feed streams accordingly. The feed lines for material flows may be installed at any point of the reactor or the feed lines for the feed streams. These may also be used in any combination desired.

This serves to ensure accurate dosage of the olefin amount in the feed streams. In addition, it is thus possible to control the temperature in the reactor precisely. For splitting the gas flow, a device for splitting the feed stream is installed directly in the feed line for the fresh feed stream. The inventive device also comprises valves by which the supply of gas to the individual spray or injection devices in the reactor can be controlled precisely. Depending on the heating extent of the gas in the individual catalyst beds, the amount of material feed is dosed. In this way it is possible to maintain the temperature inside the reactor within the specified temperature limits.

If the feed stream is passed through more than two catalyst beds, the reactor is fitted with additional catalyst beds. Also included are the required additional feed devices for the feed streams and material flows. In such case a contrivance is claimed, in which
a pipeline conveying the feed stream splits the feed stream into three or more gas flows, and
the reactor is fitted with three or more horizontally arranged catalyst beds,
three or more pipelines entering the reactor laterally being installed to introduce the feed streams into the downward material flow so that the resulting material flow can pass through the further catalyst beds, and
the pipelines for the further feed streams are fitted with feed lines for olefin-containing material flows by which it is possible to control the olefin content in the feed stream.

The feed rate and the composition of the feed stream into the reactor are preferably controlled via the temperature as parameter. Hence temperature sensors or thermometers can be installed in any place inside the reactor. Likewise there may be heating or cooling devices in any place of the inventive contrivance, which allow for additional temperature control. It goes without saying that the inventive contrivance also comprises the required control devices, no matter if these are of electric, electronic or mechanical nature. It is also possible, however, to control the feed rate and the composition of the supplied material flow via other signals as, for example, the sulphur or olefin content of the gas or of a combination of these measured values. For this purpose, there may be measuring sensors in any place of the feed lines or inside the reactor.

The contrivance according to the invention is shown in principle in patent DE 102008059243 A1. The latter differs from the present contrivance especially by the additional pipelines for olefin-containing feed streams.

The contrivance according to the invention may further comprise other devices in any place which are required to maintain optimum operation. These may be, for example, valves, pumps, gas manifolds or gas conveying devices. These may likewise be sensors, thermometers, flow meters or analysis instruments. These may be installed in any place of the inventive contrivance.

The inventive process and the inventive contrivance allow carrying out the hydrodesulphurisation of olefin-containing gases with minor equipment and without extensive cooling or heating devices. The desulphurisation is effective so that the sulphur content of the feed stream in the subsequent gas scrubbing process can be reduced to the ppb range (ppb: parts per billion, 10⁻⁷ mole percent). The process provides for reliable and safe temperature control and handling of the process. The inventive process yields a product gas which basically still only contains hydrogen sulphide in the form of a sulphur compound.

The contrivance according to the invention is illustrated in more detail by means of a drawing, the embodiment not being limited to this drawing.

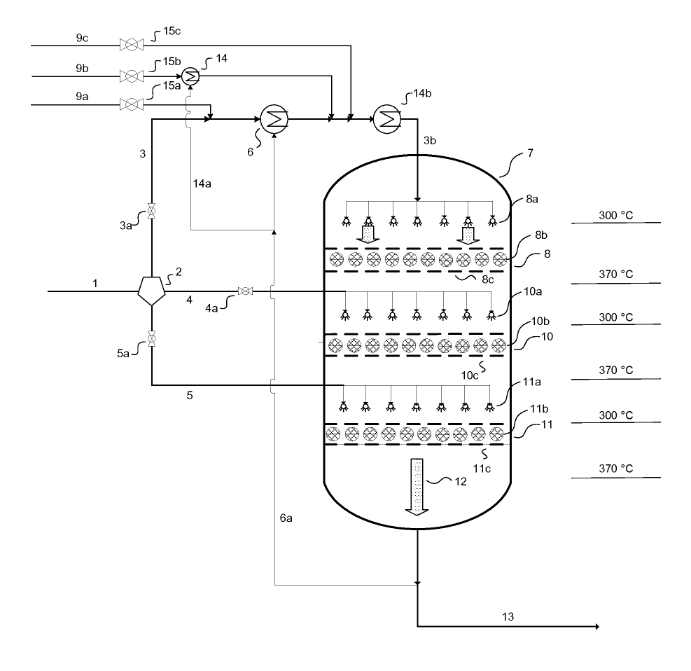

FIG. 1 shows a reactor according to the invention, fitted in an exemplary mode with three catalyst beds, for the performance of a hydrodesulphurisation. The feed stream (1) is split into three feed streams (3,4,5) by a gas manifold (2). As a rule, the feed stream already has the required olefin content. For each gas or liquid feed line, three valves (3a,4a,5a) are installed for controlling the feed stream. The first feed stream (3) is preheated by means of a heating device (6) or a heat exchanger (with heat flow, 6a) and fed (8a) to the reactor (7) via the reactor head (3b). The temperature when introducing the first feed stream is ideally 300° C. The first feed stream reaches the first catalyst bed (8) and heats up. The catalyst bed (8) contains the catalyst (8b) supported by suitable carrier particles and a grid (8c) or another suitable supporting device. The outlet temperature at the lower grid tray of the first catalyst bed (8) may be up to 390° C., however, typically is 370° C. The temperature in this first catalyst bed is controlled via the olefin content in the first feed stream (3b). As a result of a higher olefin content in the first feed stream the first catalyst bed (8) heats up more strongly. The olefin content in turn can be controlled via various material flows (9a,b,c) which, in this example, are introduced as a dilution gas flow into the first feed stream (3). This flow is an olefin-rich material flow (9a), an olefin-lean material flow (9b) or an olefin-free material flow (9c). If, for example, a feed stream (3b) of higher olefin content is required, a larger amount of the olefin-rich material flow (9a) is fed. If an olefin-leaner feed stream (3) is used, a higher amount of the olefin-lean (9b) or the olefin-free material flow (9c) is supplied. For readjustment, olefin may be re-dosed by addition of an olefin-containing material flow (9a). In this way the temperature of the first catalyst bed (8) can be controlled properly. Such procedure can also be applied to the other feed streams (4,5). In this example, another dilution stream (4) is introduced without further control into a second feed stream (10a) downstream of the first catalyst bed (8). This will make the material flow cool down again, in the ideal case to 300° C. This flow will thus reach the second catalyst bed (10) with catalyst (10b) on a supporting device (10c). Here the material flow will heat up again owing to the hydrogenation reaction. In order to set the proper reaction temperature, another feed stream (11a) is introduced downstream of the catalyst bed. The resulting material flow reaches a third catalyst bed (11) with catalyst (11b). The catalyst is supported by grids (8c,10c,11c) or other supporting devices inside the reactor. At the outlet of the reactor a product gas (12) is obtained which basically still only contains hydrogen sulphide in the form of a sulphur compound. The product gas is discharged at the outlet of the reactor (13). In this example, the first feed stream (3b) is preheated by the heat energy of the feed stream (6a) via a heat exchanger (6). The heat energy of the feed stream (13) is also used (14a) in this example to preheat the olefin-lean material flow (9b) via a heat exchanger (14), the material flow being introduced into the first feed stream (3). The feed stream (3) may be further heated via a further heat exchanger (14b) for adjusting the temperature. The individual material flows (9a,b,c) can be controlled via valves (15a,b,c). Typical reactor temperatures are specified at the side.

### LIST OF REFERENCE NUMBERS AND DESIGNATIONS

1 Feed stream (olefin-containing)
2 Gas manifold
3 First feed stream
3a Valve for controlling the first feed stream
3b First feed stream introduced via the reactor head
4 Second feed stream
4a Valve for controlling the second feed stream
5 Third feed stream
5a Valve for controlling the third feed stream
6 Heat exchanger for heating the first feed stream
6a Heat flow from feed stream for heating the first feed stream
7 Reactor
8 First catalyst bed
8a Gas feed devices for the first feed stream
8b Catalyst particles in the first catalyst bed
8c Supporting device for the first catalyst bed
9a Olefin-rich material flow
9b Olefin-lean material flow
9c Olefin-free material flow
10 Second catalyst bed
10a Gas feed devices for the second feed stream
10b Catalyst particles in the second catalyst bed
10c Supporting device for the second catalyst bed
11 Third catalyst bed
11a Gas feed devices for the third feed stream
11b Catalyst particles in the third catalyst bed
11c Supporting device for the third catalyst bed
12 Product gas
13 Product gas outlet
14 Heat exchanger for heating the olefin-lean material flow
14a Heat flow from the feed stream for heating a material flow
14b Heat exchanger for heating the first feed stream

1. Process for the desulphurisation of olefin-containing feedstocks by controlling the olefin content, comprising:
a gaseous feed stream containing olefins and hydrogen is passed through a reactor provided with a catalyst suitable for hydrodesulphurisation, and the organic sulphur compounds and olefins contained in the feed stream incorporating olefins and hydrogen are hydrogenated completely or partly into hydrogen sulphide and alkanes;
the olefin-containing feed stream is split prior to being introduced into the reactor so that at least two feed streams are formed;
the first feed stream is passed by means of suitable devices via the reactor head and through a catalyst bed inside the reactor containing a partial amount of a catalyst suitable for hydrodesulphurization; and
a second feed stream is added into the reactor laterally downstream of the first catalyst bed and to the reaction mixture heated by the first hydrogenation, and the gas flow thus obtained is passed through a second catalyst bed inside the reactor, wherein;
the content of olefins in at least one feed stream can be controlled by separately adding olefins or dilution gas to the individual feed streams, and
the temperature inside the reactor being controlled by adjusting the content of olefins in at least one feed stream.

2. Process for the desulphurisation of olefin-containing feedstocks by controlling the olefin content according to claim 1, wherein the olefin content in the first olefin-containing feed stream is adjusted by adding an olefin-lean dilution stream or an olefin-free dilution stream or both dilution streams to the first feed stream.

3. Process for the desulphurisation of olefin-containing feedstocks by controlling the olefin content according to claim 1, wherein the content of olefins in the first feed stream is increased by adding an olefin-rich material flow separately to the first feed stream.

4. Process for the desulphurisation of olefin-containing feedstocks by controlling the olefin content according to claim 1, wherein the content of olefins in the first feed stream is controlled by adding an olefin-rich dilution stream, an olefin-lean or an olefin-free dilution stream separately to the first feed stream.

5. Process for the desulphurisation of olefin-containing feedstocks by controlling the olefin content according to claim 1, wherein downstream of the second catalyst bed a third feed stream is fed into the reactor laterally downstream of the second catalyst bed to the material flow heated by the second hydrogenation, and the material flow to be hydrogenated passes the second catalyst bed first and then a third catalyst bed.

6. Process for the desulphurisation of olefin-containing feedstocks by controlling the olefin content according to claim 1, wherein the material flow, which has been sent through the third partial amount of the hydrodesulphurisation catalyst, is passed through one or several additional partial amounts of a hydrodesulphurisation catalyst and a further feed stream is introduced into the reactor laterally downstream of the catalyst beds.

7. Process for the desulphurisation of olefin-containing feedstocks by controlling the olefin content according to claim 6, wherein the olefin content in the feed streams is controlled by adding an olefin-lean material flow or an olefin-free material flow or a combination of such material flows to the feed streams.

8. Process for the desulphurisation of olefin-containing feedstocks by controlling the olefin content according to claim 1, wherein the olefin-free or olefin-lean material flow is a hydrogenous material flow.

9. Process for the desulphurisation of olefin-containing feedstocks by controlling the olefin content according to claim 1, wherein the olefin-lean or olefin-free material flow is a material flow containing methane.

10. Process for the desulphurisation of olefin-containing feedstocks by controlling the olefin content according to claim 1, wherein the olefin-lean or olefin-free material flow is a material flow containing hydrogen and methane.

11. Process for the desulphurisation of olefin-containing feedstocks by controlling the olefin content according to claim 1, wherein the first feed stream introduced at the head of the reactor is preheated.

12. Process for the desulphurisation of olefin-containing feedstocks by controlling the olefin content according to claim 1, wherein the proportion of the first gas flow introduced at the head of the reactor ranges between 1 and 99 percent by mass of the total feed stream.

13. Process for the desulphurisation of olefin-containing feedstocks by controlling the olefin content according to claim 1, wherein the proportion of the first gas flow introduced at the head of the reactor ranges between 5 and 15 percent by mass of the total feed stream.

14. Process for the desulphurisation of olefin-containing feedstocks by controlling the olefin content according to claim 1, wherein as feed stream for the hydrodesulphurisation a gas is used, major part of which contains olefins with 2 to 6 carbon atoms.

15. Process for the desulphurisation of olefin-containing feedstocks by controlling the olefin content according to claim 1, wherein as feed stream for the hydrodesulphurisation a gas is used, major part of which contains higher olefins.

16. Process for the desulphurisation of olefin-containing feedstocks by controlling the olefin content according to claim 1, wherein the hydrodesulphurisation is carried out at a temperature of 150 to 500° C.

17. Process for the desulphurisation of olefin-containing feedstocks by controlling the olefin content according to claim 1, wherein the feed stream is introduced into the reactor at a temperature of 200 to 400° C.

18. Process for the desulphurisation of olefin-containing feedstocks by controlling the olefin content according to claim 1, wherein the feed stream is introduced into the reactor at a temperature of 250 to 350° C.

19. Process for the desulphurisation of olefin-containing feedstocks by controlling the olefin content according to claim 1, wherein the hydrodesulphurisation is carried out at a pressure of 0.1 to 10 MPa.

20. Process for the desulphurisation of olefin-containing feedstocks by controlling the olefin content according to claim 1, wherein heating of the feed stream can be performed in any place desired by heat exchange with the hydrogenated feed stream.

21. Process for the desulphurisation of olefin-containing feedstocks by controlling the olefin content according to claim 1, wherein heating of an olefin-rich material flow, an olefin-lean material flow or an olefin-free material flow can be performed in any place desired by heat exchange with the hydrogenated feed stream.

22. Process for the desulphurisation of olefin-containing feedstocks by controlling the olefin content according to claim 1, wherein the hydrodesulphurisation process is followed by a gas scrubbing process or a separation of hydrogen sulphide.

23. Process for the desulphurisation of olefin-containing feedstocks by controlling the olefin content according to claim 1, wherein the hydrodesulphurisation process is followed by an adsorption process using a chemical adsorbent.

24. Contrivance for the desulphurisation of olefin-containing feedstocks by controlling the olefin content, wherein
a pipeline conveying the feed stream splits the feed stream into at least two gas flows, and
the pipeline conveying the first feed stream runs to the head of a reactor fitted with several horizontally arranged catalyst beds, the reactor having at least two horizontally arranged catalyst beds, and
a second pipeline entering the reactor laterally is installed between the first and the second catalyst bed to introduce the second feed stream into the downward gas flow so that the resulting material flow passes through the second catalyst bed, and
the pipelines for at least one feed stream are fitted with feed lines for olefin-containing material flows by which it is possible to control the olefin content in the feed stream.

25. Contrivance for the desulphurisation of olefin-containing feedstocks by controlling the olefin content according to claim 24, wherein
a pipeline conveying the feed stream splits the feed stream into further gas flows, and
the reactor is fitted with further horizontally arranged catalyst beds,
further pipelines entering the reactor laterally being installed to introduce the feed streams into the downward gas flow so that the resulting material flow can pass through the further catalyst beds, and
the pipelines for the further feed streams are fitted with feed lines for olefin-containing material flows by which it is possible to control the olefin content in the feed stream.

26. Contrivance for the desulphurisation of olefin-containing feedstocks by controlling the olefin content according to claim 25, wherein the pipeline for the first feed mixture is provided with a heating device upstream of the reactor.

27. Contrivance for the desulphurisation of olefin-containing feedstocks by controlling the olefin content according to claim 26, wherein the device for heating the first feed stream is a heat exchanger which heats the feed stream with the product gas.
